(19) United States
(12) Patent Application Publication
(10) Pub. No.: US 2006/0142602 A1
(43) Pub. Date: Jun. 29, 2006

(54) Method of making methyl ester surfactants

(76) Inventors: John Christoher Hutchinson, Chicago, IL (US); Jungsik Lee, Wilmette, IL (US); Timothy M Eigley, Estates, IL (US); Robert A Krueger, Chicago, IL (US)

Correspondence Address: McAndrews Held & Malloy, 500 W Madison Street, 34th Floor, chicago, IL, 60661, US

(21) Appl. No.: 10/534,244

(22) PCT Filed: Nov. 21, 2003
(86) PCT No.: PCT/US03/37411

§ 371 Date: Aug. 25, 2005

Related U.S. Application Data
(60) Provisional application No. 60/429,630, filed on Nov. 27, 2002.

(51) Int. Cl. C07C 67/02

(52) U.S. Cl. 554/174; 560/263

(57) ABSTRACT

A method of making a methyl ester based surfactants is provided. The method includes the transesterfication or esterification of a methyl ester feedstock and a poly(alkyl ether) feedstock.

### BACKGROUND OF THE INVENTION

The invention described herein pertains generally to a method of making methyl ester surfactants.

Surfactants have many uses including emulsion polymerization and coatings applications, hard surface cleaners, cosmetics, personal care products, laundry detergents, agricultural applications, metal working as tempering and drilling fluids. For example, alkylphenol ethoxylate (APE) surfactants are widely used in the coatings industry. APEs have been widely used and accepted because of their performance attributes such as excellent wetting properties and low pour points. APEs, however, create environmental concerns. Researchers have sought to identify a class of non-ionic surfactants which are biodegradable, economical, and have the performance attributes of APEs, such as excellent wetting properties and low pour points.

One such group of non-ionic surfactants is methyl ester (ME) based surfactants. ME-based surfactants are provided which have the excellent wetting properties and low pour points of APEs but do not implicate the environmental concerns of APES. The economics of ME-based surfactants is also attractive. The price of vegetable oil, such as coconut or palm oil, from which ME-based surfactants are made, has been competitive with the price of petroleum-based feed stocks. As petrochemical prices are expected to increase in the long run, the economics of ME-based surfactants will stay strong.

Known methods of making the ME-based surfactants, however, include the use of ethylene oxide and proprietary catalysts. That is, the starting materials are a methyl ester and the ethylene oxide monomer and the catalysts is typically an alkali/alkaline earth catalyst. These methods are not desirable for many reasons. First, the ethylene oxide starting materials are in the gas phase, highly flammable, toxic, and otherwise difficult to work with. Second, the proprietary catalysts are not readily or widely available and may be expensive to use for the general production of ME-based surfactants.

### DETAILED DESCRIPTION OF THE FIGURES

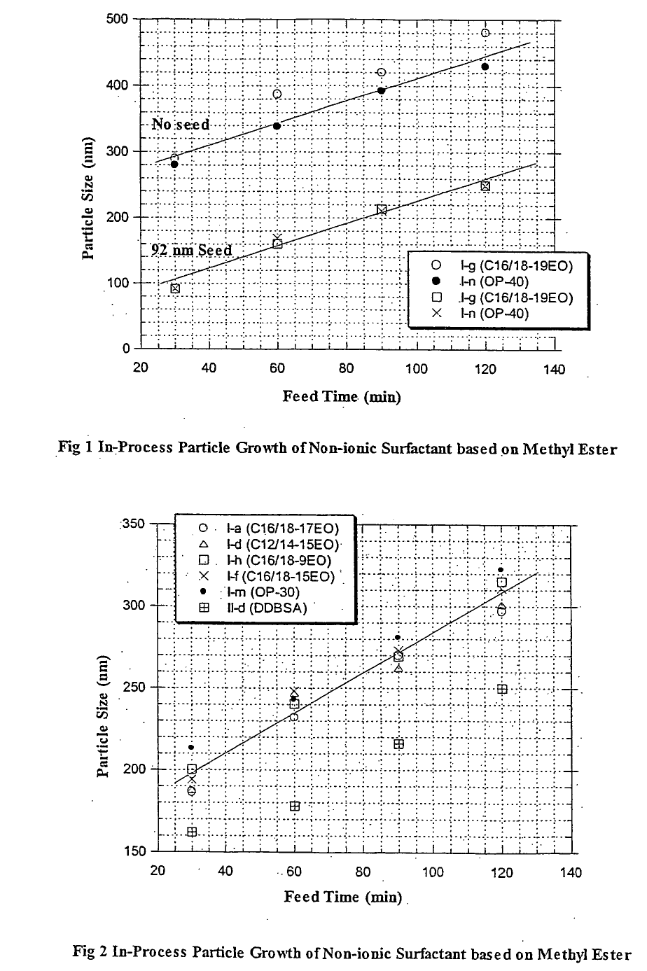

FIG. 1 illustrates the particle growth of ME-based surfactants compared with that of OP ethoxylates

FIG. 2 illustrates the particle growth of ME-based surfactants is compared with that of OP ethoxylates.

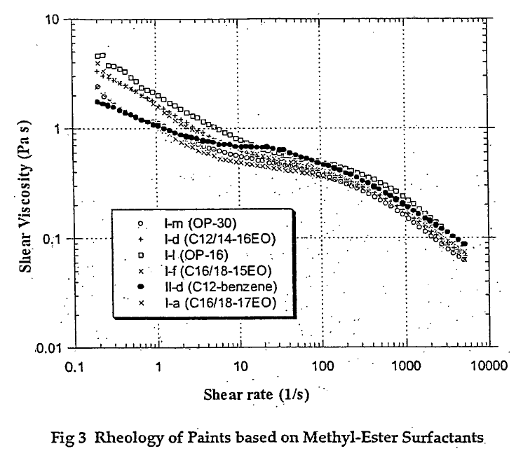

FIG. 3 illustrates the rheology of paints based on ME-based surfactants.

### SUMMARY OF THE INVENTION

In accordance with the present invention, a method of making ME-based surfactants is provided. The present invention provides a method that eliminates the use of the ethylene oxide monomer thereby eliminating the dangers associated with this highly flammable and toxic gas. Additionally, the present invention provides a method that eliminates the use of proprietary catalysts and their incumbent expenses and special handling needs. The disclosed method allows greater flexibility in controlling the alkoxy chain length distribution of the alkoxylated ester.

### DETAILED DESCRIPTION OF THE INVENTION

The method of making the ME-based surfactant of the present invention involves the transesterification of a methyl ester feedstock with a poly(alkyl ether) feedstock.

The methyl ester feedstock contains one or more of the methyl ester components listed below. Each methyl ester component has a general structure of the form R—C(O)OR′. R is an organic moiety and can be saturated or unsaturated,. branched or linear, contain from two to twenty-four carbons. R′ can be H or organic.

Those skilled in the art will understand that when R′ is H, the compound is not an ester but for simplicity of nomenclature the term methyl ester surfactant is used to also include these compounds. When R′ is H and the evolved volatile is water, the R—C(O)OR′ feedstock is known as a carboxylic acid and the reaction is formally called an esterification rather than a transesterification. The disclosed invention includes the esterification product utilizing such a feedstock.

The methyl ester feedstock may include, for example, octanoic acid (C8H16O2), decanoic acid (C12H20O2), lauric acid (C12H24O2), stearic acid (C18H36O2), methyl octanoate (C9H18O2), methyl decanate (C11H22O2), methyl laurate (C13H26O2), and methyl stearate (C19H38O2), and mixtures of these compounds.

The poly(alkyl ether) feedstock contains either a single component or a distribution of the species described below. Each poly(alkyl ether) component has general structure of the form HO(R″O)_{y}R′″. R″ is an organic moiety, can be saturated or unsaturated, branched or linear, and contain from one to six carbons. The allowed values of y range from one to 200. R′″ is either H or an organic moiety that can be saturated or unsaturated, branched or linear, and contain from one to twelve carbons. A variety of R, R′, R″, and R′″ can be present within a single poly(alkyl ether) component.

The poly(alkyl ether) feedstock may include, for example, poly(ethylene oxide), poly(ethylene oxide) monomethyl ether, poly(propylene oxide), poly(propylene oxide) monomethyl ether, poly(ethylene oxide)-block-poly(propylene oxide), random copolymers of (ethylene oxide)-(propylene oxide), and mixtures of these compounds.

Transesterification is accomplished by combining the methyl ester and poly(alkyl ether) feedstocks in the presence of a catalyst and removing the resulting volatiles. The catalyst can be (but is not limited to) H₂SO₄, NaOCH₃, or Sn(OH)C₄H₉. The catalyst is neutralized at the conclusion of the reaction to provide a stable product.

The preferred ME-based surfactants of the present invention have the following general structural formula: 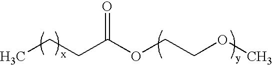
where x ranges from 0 to 22, and y ranges from 1 to 200.

The preferred ME-based surfactant is synthesized by transesterification of a methyl ester feedstock with a poly(alkyl ether) feedstock: 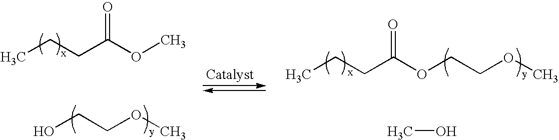
where x ranges from 0 to 22, and y ranges from 1 to 200. The catalyst can be (but is not limited to) H₂SO₄, NaOCH₃, or Sn(OH)C₄H₉. The catalyst is neutralized at the conclusion of the reaction to provide a stable product.

One typical application of ME-based surfactants is in emulsion polymerization. For emulsion polymerization applications, the desired methyl-capped poly(alkox)) chain is preferably poly(ethylene oxide) mono-methyl ether with an average molecular weight between 400 and 2000 Da. The desired methyl ester feedstock may be obtained from Stepan Company sold under the stock numbers C-48, C-65, C-66, or some combination thereof.

The following examples further set forth the method of the present invention.

### EXAMPLE I

Stepan C-65 is a methyl palmitate-oleate feed stock. Lot#137TK (used throughout this preparative method) has a saponification number of 180.25 mg KOH/g C-65 that translates as an equivalent weight of 311.23 g/mole.

Poly(ethylene glycol) monomethyl ether (MePEG750) was used as received from Aldrich Chemical Company (Lot# 14325JO), and has an average molecular weight of 762 g/mol as determined by gel permeation chromatography.

Methanolic sodium methoxide was used as received from Aldrich Chemical Company (Lot# 11106KO) and had a concentration of 0.50 M.

The temperature was regulated using a heating mantle controlled by a temperature controller-thermocouple control loop.

A three-necked one-liter glass round bottom flask was charged with 446.10 g (585.43 mmole) of MePEG750 followed by 182.20 g C-65 (585.43 mmole). The flask was then fitted with a vacuum adapter, thermocouple, and stopper. After evacuating the flask and refilling with nitrogen three times in order to minimize the amount of water and oxygen, 10 mL (0.005 moles) methanolic sodium methoxide catalyst was added by syringe. The flask was evacuated briefly, and the temperature was raised to 50° C. under static vacuum. Upon heating, the solution changed color from a faint yellow to a brownish red color. After the color change, the flask was gently evacuated and heated to 85° C. under dynamic vacuum (ca 1×10⁻³ Torr ultimate pressure) and stirred magnetically for 20 hours. The reaction was terminated after complete reaction of the starting materials by cooling to 35° C. and neutralizing the catalyst with a stoichiometric amount of glacial acetic acid.

### EXAMPLE II

Stepan C-66 is a methyl stearate feed stock. Lot#307TK (used throughout this preparative method) has an equivalent weight of 278.097 g/mole.

Poly(ethylene glycol) monomethyl ether (MePEG2000) was used as received from Aldrich Chemical Company (Lot#10329JA), and has an average molecular weight of 1960 g/mol as determined by gel permeation chromatography.

FASCAT 9100, a monobutyl tin hydroxide transesterification catalyst sold by ATOFINA, was used as received.

The temperature was regulated using a heating mantle controlled by a temperature controller-thermocouple control loop.

A three-necked one-liter glass round bottom flask fitted with a vacuum adapter, thermocouple, and stopper was purged with N₂ and charged with 406.77 g (208.19 mmole) of MePEG2000 and 51.89 g C-66 (186.59 mmole). The contents were heated to 70° C. and mixed thoroughly by magnetic stirring. When the contents had a uniform, clear colorless liquid appearance, 32 mg of the FASCAT catalyst was added under a N₂ purge and the flask evacuated. The reaction was heated to 170° C. under dynamic vacuum for 140 hours. Cooling the reaction to room temperature yielded a solid product.

### EXAMPLE III

Stepan C-65 is a methyl palmitate-oleate feed stock. Lot#137TK (used throughout this preparative method) has a saponification number of 180.25 mg KOH/g C-65 that translates as an equivalent weight of 311.23 g/mole.

Poly(ethylene glycol) monomethyl ether (MePEG550) was used as received from Aldrich Chemical Company (Lot#15714KR), and has an average molecular weight of 530 g/mol as determined by gel permeation chromatography.

Methanolic sodium methoxide was used as received from Aldrich Chemical Company (Lot#11106K0) and had a concentration of 0.50 M.

The temperature was regulated using a heating mantle controlled by a temperature controller-thermocouple control loop.

A three-necked one-liter glass round bottom flask was charged with 240.94 g (438.07 mmole) of MePEG550 followed by 136.34 g C-65 (438.07 mmole). The flask was then fitted with a vacuum adapter, thermocouple, and stopper. After evacuating the flask and refilling with nitrogen three times in order to minimize the amount of water and oxygen, 40 mL (0.020 moles) methanolic sodium methoxide catalyst was added by syringe. The flask was evacuated briefly, and the temperature was raised to 33° C. under static vacuum. Upon heating, the solution changed color from a faint yellow to a brownish red color. After the color change, the flask was gently evacuated and heated to 85° C. under dynamic vacuum (ca 1×10⁻³ Torr ultimate pressure) and stirred magnetically for 20 hours. The reaction was terminated after complete reaction of the starting materials by cooling to 35° C. and neutralizing the catalyst with a stoichiometric amount of glacial acetic acid.

### EXAMPLE IV

Stepan C-66 is a methyl stearate feed stock. Lot#307TK (used throughout this preparative method) has an equivalent weight of 278.097 g/mole.

Poly(ethylene glycol) monomethyl ether (MePEG550) was used as received from Aldrich Chemical Company (Lot#15714KR), and has an average molecular weight of 530 g/mol as determined by gel permeation chromatography.

Methanolic sodium methoxide was used as received from Aldrich Chemical Company (Lot#1 1 106KO) and had a concentration of 0.50 M.

The temperature was regulated using a heating mantle controlled by a temperature controller-thermocouple control loop.

A three-necked one-liter glass round bottom flask fitted with a vacuum adapter, thermocouple, and stopper was purged with N₂ and charged with 199.25 g (375.96 mmole) of MePEG550 and 104.54 g C-66 (375.91 mmole). The contents were heated to 35° C. and mixed thoroughly by magnetic stirring. When the contents had a uniform, clear colorless liquid appearance, 30 mL (0.015 moles) methanolic sodium methoxide catalyst was added by syringe. The flask was evacuated briefly, and the temperature was raised to 65° C. under dynamic vacuum. Upon heating, the solution changed color from a faint yellow to a brownish red color. After the color change, the flask was gently evacuated and heated to 90° C. under dynamic vacuum (ca 1×10⁻³ Torr ultimate pressure) and stirred magnetically for 20 hours. The reaction was terminated after complete reaction of the starting materials by cooling to 35° C. and neutralizing the catalyst with a stoichiometric amount of glacial acetic acid.

The reaction for each of these Examples I-IV is as follows: 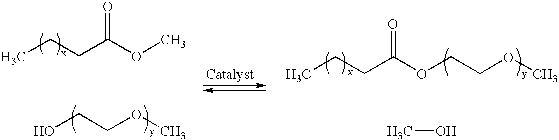
For Example I:
x=1:1 mix of 13 and 15, ester is unsaturated;
y=16.6;
sodium methoxide is the catalyst.
   For Example II:
x=1:1 mix of 13 and 15, ester is saturated;
y=43.8;
FASCAT 9100 is the catalyst.
   For Example III:
x=1:1 mix of 13 and 15, ester is unsaturated;
y=11.3;
sodium methoxide is the catalyst.
   For Example IV:
x=1:1 mix of 13 and 15, ester is saturated;
y=11.3;
sodium methoxide is the catalyst.

The method of the present invention may be used to manufacture ME-based surfactants useful in many applications. To demonstrate the utility of the ME-based surfactants made by the disclosed method, methyl ester ethoxylate was made and compared in several different applications to two known surfactants, methyl ester sulfonate and fatty amide ethoxylate. The formulas for these three compounds are as follows:

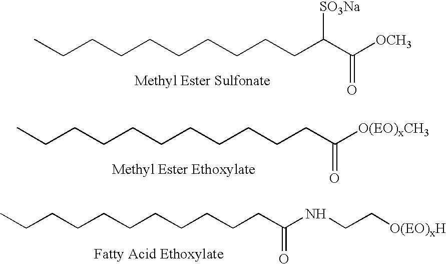

These surfactants were synthesized by the method of the present invention. Table 1a and 1b provide a detailed description of the surfactants. Octyl Phenol-based surfactants (I-k through I-n) were control samples for the evaluation of the Methyl Ester Ethoxylates and Fatty Amide Ethoxylates while Alkylbenzene Sulfonate was used as reference for the evaluation of Methyl Ester Sulfonates. The surfactants have backbone carbon chain lengths from C12/14 to C16/18 and ethylene oxide units from 6 to 22. It is noted here that ME-based surfactants with higher carbon chain, C16/18, were found to be adequate as well as their C12/14 analogs.

TABLE 1a
Surfactant Description (Non-ionic)

| | Hydrophobe | | Moles of | |
|---|---|---|---|---|
| Sample# | C-chain | unsaturation | EO | Description |
| I-a | C16/18 | y | 16.6 | Ethoxylated methyl ester |
| I-b | C16/18 | y | 44.4 | Ethoxylated methyl ester |
| I-c | C12/14 | n | 7.5 | Ethoxylated fatty amide |
| I-d | C12/14 | n | 15.5 | Ethoxylated fatty amide |
| I-e | C16/18 | n | 6.1 | Ethoxylated fatty amide |
| I-f | C16/18 | n | 14.8 | Ethoxylated fatty amide |
| I-g | C16/18 | n | 19.2 | Ethoxylated fatty amide |
| I-h | C16/18 | y | 8.5 | Ethoxylated fatty amide |
| I-I | C16/18 | y | 16.5 | Ethoxylated fatty amide |
| I-j | C16/18 | y | 21.8 | Ethoxylated fatty amide |
| I-k | OP | phenyl | 10 | Octyl phenol ethoxylate |
| I-l | OP | phenyl | 16 | Octyl phenol ethoxylate |
| I-m | OP | phenyl | 30 | Octyl phenol ethoxylate |
| I-n | OP | phenyl | 40 | Octyl phenol ethoxylate |

TABLE 1b
Surfactant Description (Anionic)

| Sample# | Hydrophobe | Description |
|---|---|---|
| II-a | C12-18 | Sulfonated methyl ester |
| II-b | C16/18 | Sulfonated methyl ester |
| II-c | C16 | Sulfonated methyl ester |
| II-d | C12-benzene | Branched alkylbenzene sulfonate |

In Table 2a and 2b, CMC and surface tension data are listed. For surface tension data the analysis was done by Wilhelmy plate method using a Krüass K-12 tensiometer. The temperature of the surface tension measurement was controlled using a Lauda-Brinkmann RM6 bath set at 25C. The critical micelle concentration was determined by plotting surface tension as a function of concentration using the Krüss K122 program. From the slope of the surface tension (γ) vs. concentration plot, surface excess (or the effectiveness of adsorption (Γ) and the area per molecule were calculated using the Gibbs equation Γ=−(1/RT)(dγ/dlnc)_{T}. The data in Table 2a indicates that ME-based surfactants have the characteristics of OP ethox-ylates with EO units longer than those of ME-based surfactants. While OP's with 30-40 EO units have surface tension of 38-45, ME-based surfactants with about 16 EO units have the same surface tension. The area per molecule and adsorption effectiveness also show a similar trend.

TABLE 2a
Surface Tension Of Non-Ionic Surfactants Based On Methyl Ester

| | | | | | | | Effec- |
|---|---|---|---|---|---|---|---|
| | | | | | | | tiveness |
| | | | | | | Area/ | of Ad- |
| | | | | | γ at | Mole- | sorption |
| | Hydrophobe | | | | CMC | cule | Γ (mol/ |
| Sam- | C- | unsatu- | Moles | CMC | (dyne/ | aₛ | cm² × |
| ple# | chain | ration | of EO | (mg/L) | cm) | (Å²) | 10¹⁰) |
| I-a | C16/18 | y | 16.6 | 7 | 39 | 84.82 | 1.958 |
| I-d | C12/14 | n | 15.5 | 37 | 38 | 59.88 | 2.773 |
| I-e | C16/18 | n | 6.1 | 13 | 37 | 51.76 | 3.208 |
| I-f | C16/18 | n | 14.8 | 21 | 45 | 88.32 | 1.88 |
| I-g | C16/18 | n | 19.2 | 72 | 46 | 137.01 | 1.212 |
| I-h | C16/18 | y | 8.5 | 17 | 42 | 72.13 | 2.302 |
| I-I | C16/18 | y | 16.5 | 45 | 44 | 103.92 | 1.598 |
| I-j | C16/18 | y | 21.8 | 66 | 46 | 122.19 | 1.359 |
| I-m | OP | phenyl | 30 | 132 | 38 | 49.92 | 3.327 |
| I-n | OP | phenyl | 40 | 158 | 45 | 89.37 | 1.858 |

TABLE 2b
Surface Tension Of Anionic Surfactants Based On Methyl Ester

| | | | | Effectiveness of |
|---|---|---|---|---|
| | CMC | γ at CMC | Area/Molecule | Adsorption Γ |
| Sample# | (mg/L) | (dyne/cm) | aₛ (Å²) | (mol/cm² × 10¹⁰) |
| II-a | 120 | 31 | 60.4 | 2.75 |
| II-b | 288 | 26 | 63.7 | 2.608 |
| II-c | 100 | 29 | 63.5 | 2.62 |
| II-d | 1135 | 35 | 75.69 | 2.194 |

In Table 3, data of interfacial tension at monomer (butyl acrylate, styrene and vinyl acetate)—water interface are shown. Interfacial tensions were determined by the pendant drop method using a Krüss Drop Shape Analysis System DSA10. The system was 0.1 wt % surfactant in water. A drop of the monomer is formed on the end of capillary tip, which is pointed downward within water phase, and its mean curvature is calculated from a digital imaging. Then the interfacial tension is deduced from LaPlace equation ΔP=(1/r₁+1/r2) 2 σ where r1 and r2 are the principal radii of curvature, ΔP the pressure difference and a interfacial tension. In the case of all three monomers, the trend is consistent with the previous observation with an exception: Fatty Amide Ethoxylates have the characteristics of OP ethox-ylates with longer EO units. OP-30 and Fatty Arnide Ethoxylates with 15-16 EO units bring the interfacial tension down to 5-6 mN/m while Methyl Ester Ethoxylates require 44 EO units to have the same effect. It is noted that Branched Alkyl Benzene Sulfonate is far more efficient than the non-ionic surfactants.

TABLE 3
Interfacial Tension of Surfactants based on Methyl Ester

| | | | | IFT | IFT | IFT |
|---|---|---|---|---|---|---|
| | | Unsatu- | Moles | (mN/m) | (mN/m) | (mN/m) |
| Surfactant | Hydrophobe | ration | of EO | BA | Styrene | VA |
| I-a | C16/18 | y | 16.6 | 9.46 | 14.01 | 9.04 |
| I-b | C16/18 | y | 44.4 | 6.46 | 6.85 | 5.02 |
| I-c | C12/14 | n | 7.5 | 6.93 | 10.79 | 8.37 |
| I-d | C12/14 | n | 15.5 | 5.58 | 6.59 | 5.41 |
| I-e | C16/18 | n | 6.1 | 8.72 | 12.28 | 8.52 |
| I-f | C16/18 | n | 14.8 | 5.59 | 6.60 | 5.23 |
| I-g | C16/18 | n | 19.2 | 6.16 | 6.11 | 5.22 |
| I-h | C16/18 | y | 8.5 | 7.45 | 11.47 | 8.74 |
| I-i | C16/18 | y | 16.5 | 5.48 | 6.15 | 5.02 |
| I-j | C16/18 | y | 21.8 | 6.21 | 6.20 | 5.27 |
| I-k | OP | phenyl | 10 | 10.12 | 14.41 | 9.93 |
| I-m | OP | phenyl | 30 | 6.25 | 6.14 | 4.83 |
| II-d | C12-benzene | | 0 | 2.90 | 0.79 | 4.41 |

ME-based surfactants made by the present method have been found to be particularly effective in emulsion polymerization. Emulsion polymerization of 35MMA/64BA/1MAA was conducted using the following as the base formula.

| | | Wt(g) | % BOM |
|---|---|---|---|
| Kettle | Water | 375 | |
| | Seed Latex* | 22.5 | 1 |
| | NaHCO3 | 0.56 | 0.08 |
| | Ammonium persulfate | 1.75 | 0.25 |
| | Water | 20 | |
| Monomer Emulsion | Water | 150 | |
| | Surfactant | 35 | 1 |
| | MMA | 245 | 35 |
| | BA | 448 | 64 |
| | MAA | 7 | 1 |
| Cofeed | Ammonium persulfate | 1.75 | 0.25 |
| | Water | 100 | |
| Rinse water | | 30 | |
| Total | | 1437 | |

Feed Time: 120 min
Temperature: 83° C.
*Seed latex: typically 50MMA/49BA/1MAA, 67 nm.

In-process particle growth was monitored. In FIGS. 1 and 2, the particle growth of ME-based surfactants is compared with that of OP ethoxylates. As shown in FIG. 1, Fatty Amide Ethoxylate with 19EO (I-g) and OP-40 have identical particle growth pattern when 92 nm seed was used. When used as the sole surfactant without seed, I-g produced larger final particle size (481 nm vs. 430 nm). In FIG. 2, various ME-based surfactants with EO units 17 or less seem to follow about the same particle growth pattern as OP-30.

In Table 4a and 4b, complete characterization data of the emulsion polymers are given. The non-ionic systems (I-a through I-m) have about the same final particle size -300 nm. The polymers were about 50% solids and free of coagulum except I-h. The lack of freeze-thaw stability was probably due to the softness of the backbone polymer (Tg=−10.7° C.). All non-ionic surfactants provided Ca²⁺ ion stability while all anionics failed. Overall performance of ME-based surfactants is similar to that of OP-30 or OP-40.

TABLE 4a
Performance of Methyl-Ester based Surfactants
in Emulsion Polymerization

| | | | | Coagu- | Particle | Vis- | |
|---|---|---|---|---|---|---|---|
| | | Moles | Solids | lum | Size | cosity | |
| | C-chain | of EO | (%) | (BOT) | (mm) | (cPS) | pH |
| I-a | C16/18 | 16.6 | 49.67 | 0.08% | 297(69) | 20 | 8.01 |
| I-d | C12/14 | 15.5 | 49.69 | 0 | 301(16) | 21.9 | 8.03 |
| I-f | C16/18 | 14.8 | 49.05 | 0 | 311(38) | 24.7 | 8.03 |
| I-h | C16/18 | 8.5 | 50.29 | 1% | 315(60) | 21.9 | 8.02 |
| I-l | OP | 16 | 50.31 | 0 | 255(51) | 15 | 8.02 |
| I-m | OP | 30 | 50.02 | 0 | 322(30) | 31.7 | 7.99 |
| II-a | C12-18 | 0 | 51.62 | 0 | 178(14) | 55.2 | 8.04 |
| II-b | C16/18 | 0 | 52.65 | 0 | 196(31) | 188.3 | 7.99 |
| II-c | C16 | 0 | 52.16 | 0 | 185(16) | 157 | 8.02 |
| II-d | DDBSA | 0 | 50.72 | 0 | 250(23) | 161.5 | 8.03 |

TABLE 4b
Performance Of Methyl-Ester Based Surfactants
In Emulsion Polymerization

| | | | Free- | Mechanical | Ca²⁺ | Heat |
|---|---|---|---|---|---|---|
| | | | thaw | Stability | Stability | Stability |
| | | Moles | Stability | (min to | (g 10% | (120 F. |
| | C-chain | of EO | (cycles) | fail) | solution) | 10 days) |
| I-a | C16/18 | 16.6 | <1 | 4:58 | 24.03 | pass |
| I-d | C12/14 | 15.5 | <1 | 6:31 | 22.31 | pass |
| I-d | C16/18 | 14.8 | <1 | 3:31 | 22.4 | pass |
| I-h | C16/18 | 8.5 | <1 | 3:34 | 20.84 | pass |
| I-l | OP | 16 | <1 | 4:10 | 21.29 | pass |
| I-m | OP | 30 | <1 | 6:39 | 20.81 | pass |
| II-a | C12-18 | 0 | <1 | 2:57 | 8.53 fail | pass |
| II-b | C16/18 | 0 | <1 | 1:47 | 5.56 fail | pass |
| II-c | C16 | 0 | <1 | 3:01 | 3.13 fail | pass |
| II-d | DDBSA | 0 | <1 | >10 (pass) | 3.7 fail | pass |

ME-based surfactants made by the present method have been found to be particularly effective in paint formulations. A simplified paint formulation with 24PVC was prepared. DuPont R-942 TiO2 slurry was mixed with other ingredients including latex binder and Rohm and Haas rheology modifiers RM-2020 and RM-825.

| | |
|---|---|
| R-942 pigment | 72.4 |
| Propylene Glycol | 13.2 |
| Tamol 731A | 1.4 |
| Latex | 105 |
| RM-2020 | 3.8 |
| RM-825 | 0.4 |
| Water | 18.4 |
| Total | 214.6 |

The amount of the Rohm and Haas rheology modifiers was adjusted for the paint viscosity of 1000 cPs by Bookfield viscometer. Table 5 shows the amounts.

TABLE 5
Thickener Demand in the Paint Formulation
of Methyl-Ester based Surfactants

| | | | | | Brookfield |
|---|---|---|---|---|---|
| | | | Thickener demand | | Viscosity, |
| | Surfactant | | (% on total) | | spindle 4, |
| Sample # | Hydrophobe | #EO | RM-2020 | RM-825 | 60 rpm |
| I-a | C16/18 | 16.6 | 1.85 | 0.56 | 937 |
| I-d | C12/14 | 15.5 | 1.85 | 0.45 | 950 |
| I-f | C16/18 | 14.8 | 1.84 | 0.64 | 1030 |
| I-l | Octylphenol | 16 | 1.83 | 0.5 | 1110 |
| I-m | Octylphenol | 30 | 1.83 | 0.6 | 930 |
| II-d | C12-benzene | 0 | 1.85 | 0.21 | 1077 |

The paint rheology profile, shown in FIG. 3, is uniform. The paint made with Alkylbenzene sulfonate latex (filled circle) appears to be more Newtonian probably due to the smaller particle size (250 nm vs. 300 nm). All other paints of non-ionic surfactant latices have similar shear-thinning behavior.

The invention has been described with reference to preferred and alternate embodiments. Modifications and alterations will occur to others upon the reading and understanding of the specification. It is intended to include all such modifications and alterations insofar as they come within the scope of the appended claims or equivalents thereof.

1: A method of making a methyl ester based surfactant comprising the steps of:
(a) providing a methyl ester feedstock comprising a methyl ester component having a general structure of the form R—C(O)OR′, wherein R is an organic moiety and can be saturated or unsaturated, branched or linear, contain from about two to about twenty-four carbons, and wherein R′ can be H or an organic moiety;
(b) providing a poly(alkyl ether) feedstock comprising a poly(alkyl ether) component having a general structure of the form HO(R′ O)_{y}R′″, wherein R″ is an organic moiety, can be saturated or unsaturated, branched or linear, and containing from about one to about six carbons, wherein y ranges from about one to about 200, and wherein R′″ is either H or an organic moiety that can be saturated or unsaturated, branched or linear, and contains from about one to about twelve carbons; and
(c) reacting the methyl ester feedstock with the poly(alkyl ether) feedstock.

2: (canceled)

3: (canceled)

4: The method of claim 1, further comprising the step of providing a reaction catalyst.

5: The method of claim 4, wherein the reaction catalyst is selected from the group consisting of acid catalysts, base catalysts or organomewallic catalysts.

6: The method of claim 4, wherein the reaction catalyst is selected from the group consisting of H₂SO₄, NaOCH₃, and Sn(OH)C₄H₉.

7: The method of claim 1, wherein the methyl ester feedstock has the following structural formula: 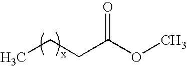
where x is 0 to 22.

8: The method of claim 1, wherein the methyl ester feedstock is a methyl ester derived from a fatty acid having between 8 and 24 carbon atoms.

9: The method of claim 1, wherein the methyl ester feedstock is selected from the group consisting of octanoic acid, decanoic acid, lauric acid, stearic acid, methyl octanoate, methyl decanate, methyl laurate, methyl stearate, and methyl palminate-oleate.

10: The method of claim 1, wherein the poly(alkyl ether) feedstock has the following structural formula: 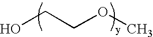
where y is from 1 to 200.

11: The method of claim 1, wherein the poly(alkyl ether) feedstock is selected from the group consisting of poly(ethylene glycol) monomethyl ether, poly(ethylene oxide), poly(ethylene oxide) monomethyl ethers poly(propylene oxide), poly(propylene oxide) monomethyl ether, poly(ethylene oxide)-block-poly(propylene oxide), random copolymers of (ethylene oxide)-(propylene oxide), and mixtures thereof.

12: The method of claim 1, wherein the reaction is carried out at a temperature between about 50° C. and about 170° C.

13: The method of claim 1, wherein the method further comprises the step of removing reaction-by-products as the reaction proceeds

14: A method of making a methyl ester based surfactant comprising:
(a) providing a methyl ester feedstock comprising a fatty acid or fatty acid derived methyl ester;
(b) providing a poly(alkyl other) feedstock; and
(c) reacting the methyl ester feedstock and poly(alkyl ether) feedstock in the presence of a reaction catalyst.

15: The method of claim 14, wherein the reaction catalyst is selected from the group consisting of acid catalysts, base catalysts or organo-metallic catalysts.

16: The method of claim 14, wherein the catalyst is selected from the group consisting of H₂SO₄, NaOH₃, and Sn(OH)C₄H₉.

17: The method of claim 14, wherein the methyl ester feedstock is a methyl ester derived from a fatty acid having from about 2 to 22 carbon atoms.

18: The method of claim 14, wherein the methyl ester feedstock is selected from the group consisting of octanoic acid, decanoic acid, lauric acid, stearic acid, methyl octanoate, methyl decanate, methyl laurate, methyl stearate, and methyl palminate-oleate.

19: The method of claim 14, wherein the poly(alkyl ether) feedstock is selected from the group consisting of poly(ethylene glycol) monomethyl ether, poly(ethylene oxide), poly(ethylene oxide) monomethyl ether, poly(propylene oxide), poly(propylene oxide) monomethyl ether, poly(ethylene oxide)-block-poly(propylene oxide), random copolymers of (ethylene oxide)-(propylene oxide), and mixtures thereof.

20: The method of claim 14, wherein the method further comprises the step of removing reaction by-products as the reaction proceeds.

21: A methyl ester based surfactant made according to the method of claim 1.

22: The methyl ester based surfactant of claim 21, wherein the methyl ester based surfactant has the following structural formula: 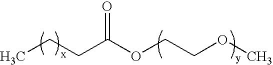
where x ranges from about 0 to about 22, and y ranges from about 1 to about 200.

23: The methyl ester based surfactant of claim 21, wherein x is between about 10 and about 20 carbon atoms.

24: A methyl ester based surfactant made according to the process of claim 16.
